(19) United States
(12) Patent Application Publication
(10) Pub. No.: US 2025/0387327 A1
(43) Pub. Date: Dec. 25, 2025

(54) DRY POWDER INHALER PHARMACEUTICAL COMPOSITION OF COATED CRYSTALLINE DRY POWDER FOR INHALATION

(71) Applicant: HOVIONE SCIENTIA LIMITED, Cork (IE)

(72) Inventors: Beatriz FERNANDES, Lisboa (PT); Filipe VULTOS, Forte Da Casa (PT); Eunice COSTA, Lisboa (PT)

(21) Appl. No.: 18/879,480

(22) PCT Filed: Jul. 4, 2023
(86) PCT No.: PCT/GB2023/051755

§ 371 Date: Dec. 27, 2024

(30) Foreign Application Priority Data
Jul. 4, 2022 (PT) 118081

(51) Int. Cl. A61K 9/00; A61K 9/14; A61K 9/16

(52) U.S. Cl. CPC A61K 9/0075; A61K 9/145; A61K 9/1694

(57) ABSTRACT

The present invention describes a dry powder inhaler pharmaceutical composition comprising one or more active pharmaceutical ingredients (API) coated with one or more force control agents (FCA) with optimized aerodynamic performance by micronizing crystalline API particles to the respirable range and coating the particles with a force control agent. The present invention also id pharmaceutical composition, and its use in medicine.

### TECHNICAL FIELD OF THE INVENTION

The present invention relates in general to inhalable powders and to methods for making them, and more particularly to a dry powder inhaler pharmaceutical composition comprising one or more active pharmaceutical ingredients (API) coated with one or more force control agents (FCA). The present invention also relates to a process for manufacturing a dry powder inhaler pharmaceutical composition with optimized aerodynamic performance by micronizing API particles to the respirable range and coating the particles with a force control agent. More particularly, it relates to the micronization process to generate a liquid mixture containing suspended API and dissolved FCA, followed by the spray-drying of the particle of micronized API coated by FCA. The generated pharmaceutical composition can be applied in the pharmaceutical field more specifically in high drug load inhalable powders or in insoluble active ingredients.

Dry Powder inhalers (DPIs) are commonly employed delivery systems for the pulmonary administration of active pharmaceutical ingredients (API) for the treatment of diseases such as asthma or chronic obstructive pulmonary disease. The majority of DPIs are developed as carrier-based mixtures in which a coarse, inert carrier is mixed with the micronized drug substance particles with a particle size in the respirable range (<5 μm). Addition of excipient to micronized crystalline API by solid blending is efficient in improving aerodynamic performance for low API loads, but as excipient-API interaction is saturated, API-API interaction lead to aerodynamic performance challenges. Carrier-free dry powder formulations for inhalation are also widespread solutions for delivery of active ingredients to the lungs. Nevertheless, more recently large API load formulations have received considerable attention for acute respiratory treatments such as antibiotics, antivirals, or vaccines. The delivery of large API loads to the lung can be achieved by formulations comprising high percentages of API with a good aerodynamic performance. Such solutions with a high drug load are usually characterized by highly adhesive and cohesive powders due to low median particle size (high surface area), thus the added excipient must act efficiently to promote particle dispersion upon actuation. Particle engineering by spray-drying of high load formulations from a solution can lead to good performances but tends to present a more challenging stability due to the amorphous state of API and excipients. Moreover, amorphous API can result in a faster API release which may not be suitable for a specific treatment.

Therefore, we have appreciated that a high load formulation comprising crystalline API and efficient low dosage excipient (by coating the API surface) will enable the development of novel products and thus more effective treatment.

We have found that the most relevant obstacle when developing such formulations is to overcome the inter-particulate forces between micronized drug substance particles which are typically highly cohesive leading to poor aerosolization performance. The present invention seeks to address this problem.

It has been known to introduce a force control agent (FCA) on to API particles surface by impact methods such as mechanofusion. This is a highly energetic dry coating process designed to fuse an FCA around the API surface (Begat et al., The Role of Force Control Agents in High-Dose Dry Powder Inhaler Formulations, Journal of Pharmaceutical Sciences, Vol. 98, No. 8, August 2009; Begat, P and Price, R., The Influence of Force Control Agents on the Cohesive-Adhesive Balance in Dry Powder Inhaler Formulations). In this process the particles are subjected to high shear forces and highly localized compression forces.

Impact methods for the preparation of formulations in which the active pharmaceutical ingredient (API) is combined with a FCA are disclosed in:

U.S. Pat. No. 11,103,448B2 and US20160158150A1 relate to a process in which the additive material (such as leucine as force control agent) is included in the formulation by means of co-jet milling with the API particles. Milling is also described in U.S. Pat. No. 10,022,303B2 in which the force control agent (or facilitating agent) can be part of a millable grinding matrix in a dry milling process or in which the facilitating agent (such as leucine) is added to the particles at the end of dry milling and then further processed by mechanofusion, cyclomixing, or high-pressure homogenization. U.S. Pat. No. 8,303,991B2, U.S. Pat. No. 895,661B2, U.S. Pat. No. 9,931,304B2 also describe a process in which the additive material (or FCA) is combined with the API by means of milling producing composite particles in which the FCA is preferably in the form of coating.

In comparison to these methods which can lead to uncontrolled particle modification, the present invention enables a more precise control of the size distribution of the particles without inducing modifications in the polymorphic form or inducing chemical degradation to the API during both the micronization and coating with the FCA. Additionally, by presenting the FCA dissolved in solution, the present invention allows a more controlled and uniform deposition of the agent on to the API particle surfaces potentially leading to improved coating homogeneity. A process for controlled particle size reduction within a narrow distribution by wet milling followed by isolation of the powder by spray-drying is described in U.S. Pat. No. 9,956,144B2. A preferred aspect of the present invention is that the process herein claimed leads to the production of encapsulated API particles with an FCA specifically aimed at improving the aerosolization performance of powders.

ES2548884T3 relates to a method for preparing glycopyrrolate particles combined with FCAs. The process described involves mechanofusion, cyclomixing, impact milling or milling by high pressure homogenization. The present invention has the advantage of including a more controlled and smoother micronization step of the active ingredient followed by surface coating with the FCA by spray-drying which is an innovative aspect comparing to the above mentioned patent.

The production of composite particles by spray-drying is widely known. Methods for producing encapsulated API particles involving a spray-drying step are disclosed in:

U.S. Pat. No. 8,668,934B2 describes a method comprising two different solvents in which the API and the excipient (an amino acid or phospholipid) have differentiated solubilities (excipient more soluble in the first solvent which is of higher polarity, while the API is more soluble in the less polar second solvent) followed by spray-drying to isolate the composite particles. The same patent describes a formulation comprising the API and an excipient at least partially encapsulating the API wherein the excipient is more soluble in water than the API. JP695341B2 relates to a method in which a lipophilic drug is solubilized in terpenes and then a functional excipient (e.g., leucine) is added in water forming a final emulsion that is subsequently spray-dried to isolate the dry powder.

An important aspect of the invention herein described is that in contrast to the patents mentioned above, the active agent is not spray-dried in the solution state which means that the API will keep its crystalline state upon being spray-dried and no amorphization process will occur as is typical of spray-drying processes. Therefore, the pharmaceutical compositions provided by the present invention present a higher physical stability since amorphous products have a tendency for crystallization. Relatively to the aforementioned methods, the process described in the present invention also has the advantage of being capable of operation using a single solvent for the micronization and API encapsulation steps leading to a more efficient and economic process.

JP20111019970A and WO2004093848 describe a DPI device comprising composite particles containing a FCA to improve the aerosolization performance of the active component. In contrast to the invention herein described, these documents do not describe a strategy for the controlled micronization of the API while keeping its crystalline form, or for achieving the surface coating of the active particles with the FCA. Furthermore, these applications describe the co-spray drying of the active ingredient with a force control agent which has already been described before (e.g., U.S. Pat. No. 8,668,934B2). In these cases, both the active ingredient and excipients are dissolved in the process solvent. However, in the present process, the active ingredient is not dissolved, but is suspended in the antisolvent while the FCA is dissolved in it. We have found that this leads to a particle having the active ingredient micronized in the crystalline state, coated with a force control agent having improved aerodynamic performance and stability.

WO2005025535 relates to a process for producing composite particles by spray-drying a solution or suspension containing the API and the FCA. A preferred aspect of the present invention is that the process herein described includes the controlled micronization of the crystalline API to achieve a tailored particle size suitable for the delivery of the drug to the target region in the lung.

From the prior art mentioned, none of the methods described effectively solves the challenge of producing high dosage DPI formulations by means of encapsulating crystalline API particles with FCAs.

### THE INVENTION

According to one aspect, the present invention provides a pharmaceutical composition suitable for a dry powder inhaler, which composition comprises particles comprising one or more micronized crystalline active pharmaceutical ingredients (API) coated with one or more force control agents (FCA). The composition is suitably an inhalable powder, such as a dry powder.

It will be understood that the composition will typically comprise a population of particles, such that the particle population will have a measurable particle size distribution. The particle size of the particles comprising the one or more micronized crystalline API is suitable for inhalation, for example by a dry powder inhaler (DPI).

In a further aspect, the invention provides a process for manufacturing a pharmaceutical composition, suitable for a dry powder inhaler, according to the invention claimed and defined herein, which process comprises the steps of:
a. Reducing the particle size distribution of the composition to obtain the micronized crystalline one or more API with a target particle size distribution suspended in an antisolvent system;
b. Adding one or more FCA soluble in the said antisolvent system, so as to provide a mixture of antisolvent with FCA dissolved therein, and micronized API in suspension;
c. Removal of the antisolvent from the mixture by spray drying, so as to obtain coating of the micronized crystalline API with the dissolved FCA.

The step of adding one or more FCA soluble in the said antisolvent system may be carried out either before or after, or both before and after, said reducing step. In a preferred aspect, the step of reducing the particle size distribution of the composition to obtain the micronized crystalline one or more API comprises wet milling a suspension of the crystalline API, preferably by a technique such as microfluidization or high-pressure homogenization. Preferably, jet-milling is not used.

The invention also provides a pharmaceutical composition comprising micronized coated API particles obtained or obtainable by a process according to the invention claimed and described herein.

The invention thus provides a process as described, and a pharmaceutical composition made according to such process, wherein during the process, the active ingredient (API) is not dissolved, but is suspended in an antisolvent while the one or more force control agents (FCA) is dissolved in the antisolvent. Under these conditions, the mixture may be wet milled, for example as described herein, for example using microfluidization or high-pressure homogenization to reduce the particle size distribution of the API. The mixture is subsequently spray-dried under these conditions i.e., where the API is suspended in an antisolvent while the one or more force control agents (FCA) is dissolved in the antisolvent.

In a further aspect, there is also provided a pharmaceutical composition in the form of an inhalable dry powder, which composition comprises particles comprising one or more micronized crystalline active pharmaceutical ingredients (API) coated with one or more force control agents (FCA) wherein the inhalable powder is obtained by wet polishing comprising a wet-milling step and a spray drying step. The wet-milling step preferably comprises microfluidization or high-pressure homogenization. Suitably, the wet-milling step is carried out on a suspension of the API, for example a suspension of the crystalline API in an antisolvent. This may for example be an aqueous system, such as water. The one or more FCA may be dissolved in the antisolvent (i.e. the antisolvent for the API acts as a solvent for the FCA) prior to the wet-milling step. Or the one of more FCA may be dissolved in the antisolvent after the wet-milling step.

In a further aspect, there is also provided a pharmaceutical composition in the form of an inhalable dry powder, which composition comprises particles comprising one or more micronized crystalline active pharmaceutical ingredients (API) coated with one or more force control agents (FCA) wherein the said coated particles are obtained by addition of the said one or more force control agents (FCA) to a wet-milled crystalline suspension of the API prior to spray drying. The wet-milled crystalline suspension is preferably prepared by microfluidization or high-pressure homogenization. Suitably, the wet-milling step is carried out on a suspension of the API, for example a suspension of the crystalline API in an antisolvent. This may for example be an aqueous system, such as water. The one or more FCA may be dissolved in the antisolvent (i.e. the antisolvent for the API acts as a solvent for the FCA) prior to the wet-milling step. Or the one or more FCA may be dissolved in the antisolvent after the wet-milling step. The resulting mixture may then be spray dried.

Accordingly, the coated particles of the invention may thus comprise a coating which has been formed on the API particles by wet-milling a suspension of crystalline API in an antisolvent comprising a force control agent (FCA) dissolved therein, and spray drying the resulting mixture. Preferably, the coating on the API particles is substantially uniform.

In a further aspect, the invention also provides a dry powder inhaler comprising a pharmaceutical composition according to the invention claimed and described herein.

In a further aspect, the invention also provides a pharmaceutical composition according to the invention as claimed and described herein, for use as a medicament. For example, for use in the treatment of a pulmonary condition in a human or animal patient. Administration of the medicament may be by any suitable means but is preferably via dry powder inhaler.

The present invention describes a dry powder inhaler pharmaceutical composition comprising one or more active pharmaceutical ingredients (API) coated with one or more force control agents (FCA). The one or more API is crystalline. The present invention also describes a process to manufacture crystalline high dosage dry powder formulation for inhalation with an optimized aerodynamic performance by coating the API micronized to the inhalation range with a force control agent upon spray-drying the suspension of API with dissolved force control agent. The invention addresses unwanted consequences of aerosolized API alone formulations, particularly low performance with high variability due to strong API-API particle interactions.

In one aspect, the API excludes crystalline N-{3-[(IS)-I-{[6-(3,4-dimethoxyphenyl) pyrazin-2-yl]amino}ethyl]phenyl}-5-methylpyridine-3-carboxamide (compound X). In another aspect, the pharmaceutical composition excludes a pharmaceutical composition comprising crystalline compound X (especially Form A) and leucine or comprising crystalline compound X (especially Form A) and L-leucine or comprising crystalline compound X (especially Form A) and lactose. More particularly, the pharmaceutical composition may exclude a pharmaceutical composition produced by spray drying a composition comprising 31.4 g of crystalline compound X and 0.628 g of L-leucine; or exclude a pharmaceutical composition produced by spray drying a composition comprising 31.0 g of crystalline compound X and 1.861 g of L-leucine; or exclude a pharmaceutical composition produced by spray drying a composition comprising 31.5 g of crystalline compound X and 3.152 g of L-leucine. Also more particularly, the pharmaceutical composition may exclude a pharmaceutical composition comprising 96% of crystalline compound X Form A and 4% of L-leucine (by weight). Also more particularly, the pharmaceutical composition may exclude a pharmaceutical composition produced by spray drying a composition comprising 249 g of crystalline compound X and 5.0 g of L-leucine; or exclude a pharmaceutical composition produced by spray drying a composition comprising 230 g of crystalline compound X and 4.9 of L-leucine. Also more particularly, the pharmaceutical composition may exclude a pharmaceutical composition comprising a composition formed by mixing 0.14 kg of L-leucine with a micronized suspension of 1.86 kg of crystalline compound X Form B in water (5% w/w/suspension) and spray drying the mixture.

### DETAILED DESCRIPTION OF THE INVENTION

One aspect of the present invention is a dry powder pharmaceutical composition of an active pharmaceutical ingredient (API) coated with a force control agent (FCA). The pharmaceutical composition may also include two or more excipients used to formulate the API as a bulk intermediate drug product. Another aspect of the present invention is a formulation of API coated with a force control agent, wherein the particles comprising the API and FCA have a particle size distribution within the inhalation range. The “inhalation range” as used herein is the particle size range expected to ensure delivery of the formulated particle to the airway's surfaces. Preferably, a particle size distribution with a Dv90<10 μm. Most preferably, a particle size distribution with a Dv90<6 μm.

The invention is applicable to all drug substances in the crystalline form insoluble in a solvent in which the force control agent is soluble. Examples include water insoluble APIs (for example, corticosteroids such as Fluticasone Furoate, low solubility antibiotics, antifungals such as itraconazole, low solubility antivirals such as remdesivir, antiparasitics such ivermectin; with aminoacids (water soluble) as force control agents. An example of these is presented herein.

The term “force control agent” (FCA) as used herein describes compounds which exhibit anti-adherent and/or anti-friction properties, such as amino acids or derivatives (e.g., L-leucine, tri-leucine, arginine, alanine), phospholipids (e.g., 1,2-distearoyl-sn-glycero-3-phosphocholine (DSPC), lecithin, or fatty acid derivatives (e.g., magnesium stearate).

The terms Dv10, Dv50, and Dv90 as discussed herein are known to those skilled in the art. Dv50 refers to the maximum particle diameter below which 50% of the sample volume exists. Dv90 refers to the maximum particle diameter below which 90% of the sample volume exists. Dv10 refers to the maximum particle diameter below which 10% of the sample volume exists.

One aspect of the present invention is a dry powder pharmaceutical composition of API coated with a force control agent presenting an increase of fine particle fraction when compared with the micronized API alone. A product of interest in the present invention may, for example, be a formulation of API coated with a force control agent with a mass median aerodynamic diameter which is lower compared to the micronized API alone, which is uncoated with FCA.

The term “fine particle fraction” and “mass median aerodynamic diameter” as discussed herein are known to those skilled in the art. “Fine particle fraction” refers to the fraction of API with an aerodynamic particle size diameter<5 μm. “Mass median aerodynamic diameter” refers to the diameter at which 50% of the particles of an aerosol by mass are larger and 50% are smaller. The term “aerodynamic particle size diameter” as discussed herein refers to the diameter of a spherical particle whose density is 1 g cm-3 which settles in still air at the same velocity as the particle in question. This diameter is obtained from aerodynamic classifiers such as cascade impactors.

One aspect of the present invention is a dry powder pharmaceutical composition comprising one or more APIs and one or more excipients, for example one or more FCA, and these ingredients may be present in any suitable amount. Preferably, in an example, the one or more FCA is present in a concentration of 30% w/w or lower (with respect to the mass of the API component), preferably 15% w/w or less, and most preferably 10% w/w or less.

The present invention also describes a new manufacturing process for manufacturing a dry powder inhaler formulation with controlled aerodynamic particle size distribution comprising one or more API and one or more excipients/FCAs comprising the steps described herein.

In a preferred aspect, the population of particles comprising a pharmaceutical composition according to the invention described herein has a particle size range wherein the Dv90 of is equal to or less than 10 μm. In an example, the particle size range may be such that the Dv90 is equal to or less than 6 μm.

In a preferred aspect, a pharmaceutical composition according to the invention described herein is provided in which the particles comprising said micronized crystalline one or more API coated with one or more FCA have a higher fine particle fraction (FPF) when compared with a pharmaceutical composition comprising the same micronized particles but without any FCA. In one aspect, the fine particle fraction (FPF) may be 30% or more of the emitted dose, when testing a capsule comprising the said composition in a dry powder inhaler.

In a further aspect, a pharmaceutical composition according to the invention is provided wherein the particles comprising said micronized crystalline one or more API coated with one or more FCA have a decreased variability with respect to fine particle fraction (FPF) when compared with a pharmaceutical composition comprising the same micronized particles but without any FCA. This decreased variability can, for example, be measured and assessed by considering the relative standard deviation (RSD) which applies to the FPF measurements. The invention provides much greater consistency of FPF.

In a preferred aspect of the invention, the one or more FCA may be any suitable agent which exhibits anti-adherent and/or anti-friction properties in pharmaceutical formulations comprising one or more APIs. Suitably, the FCA may be chosen from the group comprising: leucine (i.e. L-leucine, isoleucine, tri-leucine, distearoylphophatidylcholine (DSPC), dipalmitoylphosphatidylcholine (DPPC), alanine, arginine, histidine, lysine, valine, lecithin or a stearate such as magnesium stearate; or a or a combination of two or more thereof.

The FCA may be a phosphoglyceride chosen from the group comprising a phosphatidylcholine, a phosphatidylglycerol, or a phosphatidylethanolamine, or a combination of two or more thereof.

The FCA may for example be dipalmitoylphosphatidylcholine (DPPC), distearoylphosphatidylcholine (DSPC), dimyristoylphosphatidylcholine (DMPC) or lecithin, or a combination of two or more thereof.

In a preferred aspect of the invention, the one or more API employed in the pharmaceutical composition is a crystalline API which is insoluble in a solvent in which the FCA used in the composition is soluble. The one or more API may include API such as corticosteroids such as fluticasone propionate and budesonide; long-acting β adrenoceptor agonists (LABAs) such as indacaterol, vilanterol, salmeterol and formoterol; short-acting β₂ adrenergic receptor agonist (SABA) such as albuterol; long-acting, inhaled muscarinic antagonist (LAMA) such as aclidinium bromide; antipsychotics such as Loxapine; anti-Parkinson drugs such as levodopa; antibiotics such as tobramicyn; antifungals such as itraconazole or antivirals such as remdesivir and laninamivir; or antiparasitics such as ivermectin.

In one aspect of the invention, the one or more FCA is present (as a total) in an amount of 30% or less by weight of FCA per weight of total API. In an example, this amount may be 15% w FCA/w API or less; or 10% w FCA/w API or less. In a preferred aspect, the amount of FCA is at least 5% or more, or at least 7% or more, per weight of total API. A preferred range may for example be from 5% to 20%, or from 7% to 15%, per weight of total API. The amount required to achieve the desired results can vary to some extent depending upon the API, and in some cases an amount of FCA up to 50%, for example ranging from 25% to 50%, per weight of total API, may be employed.

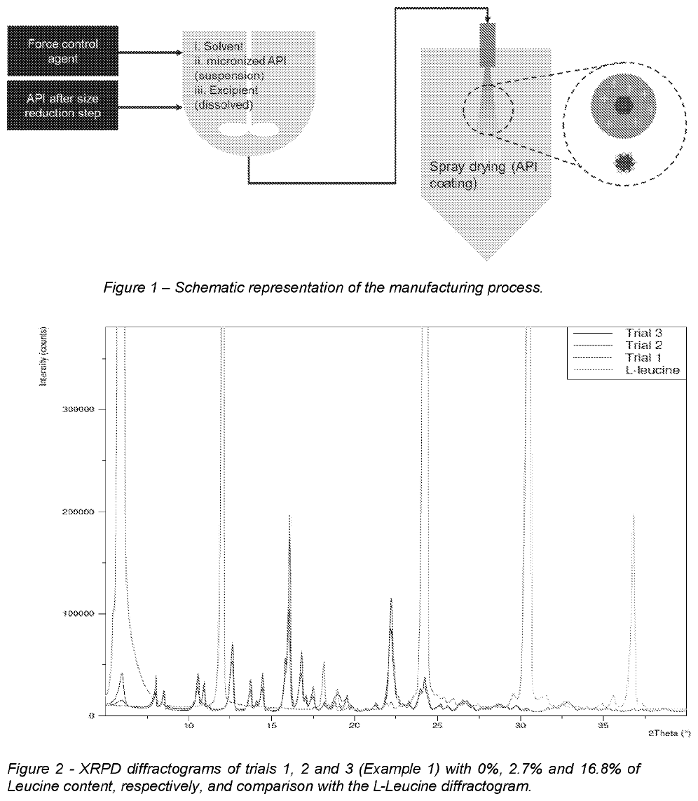

In the invention, the particles of the one or more API are not simply mixed with the one or more FCA, but are intimately coated with FCA, as for example illustrated schematically in FIG. 1. Suitably, each particle within the population of particles has a coating of FCA.

In the process of the invention, the step of reducing the particle size distribution, which step is employed to obtain micronized crystalline API with a desired or target particle size distribution, preferably comprises subjecting the particles to a wet-milling step. This may for example comprise one or more of high-pressure homogenization, microfluidization, ball milling, high shear mixing or any combination thereof. High-pressure homogenization and microfluidization are particularly preferred. As indicated above, in a preferred aspect, the population of particles comprising a pharmaceutical composition according to the invention described herein has a particle size range wherein the Dv90 of is equal to or less than 10 μm or may be such that the Dv90 is equal to or less than 6 μm.

Preferably, the one or more API remains in a crystalline state throughout the process of the invention. Suitably, the one or more API is provided in a liquid suspension, prior to drying.

In a preferred aspect, the particle size distribution reduction step comprises the use of high-pressure homogenization or microfluidization using a solvent system in which the one or more API is insoluble. That is to say, the API is in suspension, and suitably the particle size distribution reduction step is carried out on a suspension of crystalline API.

The temperature employed during the particle size distribution reduction step is preferably at or below about 60° C.; although, depending upon the API and the intensity of the process used, may be at or below about 10° C. Temperatures within this range may also be employed. For example, the temperature in the particle size distribution reduction step may be at or above about 20° C., although is still preferably below 60° C.

The pressure used during the carrying out of the particle size distribution reduction step is also a consideration. Preferably, the pressure in the particle size distribution reduction step is at or below about 100 bar, although may be at or below about 50 bar. Pressures within this range may also be employed. For example, the pressure used in the particle size distribution reduction step may be at or above about 10 bar, although in one aspect is still preferably below 100 bar. However, for some processes, the pressure in the particle size distribution reduction step may be 100 bar or greater.

In the process of the invention, preferably the antisolvent system comprises only one (that is, a single) antisolvent. The term antisolvent as used herein is with reference to the one or more API, since the feature of a suspension of API is important to the present invention. Suitably, the antisolvent will be a solvent for the one or more FCA. Use of a single solvent is a particular advantage of the present process and avoids the need for multiple or different solvents. In one preferred aspect, a single solvent is used-one in which the one or more API is insoluble, and the one or more FCA is soluble. This may for example be for all steps of the process—that is for steps (a), (b), and (c) as described and claimed herein. An antisolvent or antisolvent system is a solvent or solvent system in which the API is insoluble. For example, less than 1% of the API (by weight) will dissolve in the antisolvent. Suitable antisolvents will be apparent to those skilled in the art, depending upon the identity of the API.

Any suitable antisolvent system may be used, although preferably the antisolvent system is chosen from the group comprising: water, ethanol, methylene chloride, methanol, and other alcohols, such as a C₃, C₄ or C₅ aliphatic alcohol; a ketone; and polar protic solvents. Ketone solvents may include for example acetone, methyl ethyl ketone, or methyl isobutyl ketone. An aqueous system is preferred, especially one comprising water. This works well for APIs which are not soluble in water or polar solvents.

In the process of the invention, suitably the total solids content (referring to both undissolved and dissolved solids) in the antisolvent system is at or below about 20% by weight, preferably at or below 15% by weight, most preferably at or below 10% by weight.

Where a micronization chamber is employed in the particle size distribution reduction step, for example when using microfluidization, this preferably has an internal diameter below 500 um, preferably below 200 um, most preferably below 150 um.

The process of the invention includes removal of the antisolvent from the mixture by spray drying, so as to obtain coating of the micronized crystalline API with the dissolved FCA. The spray drying suitably comprises spray drying of a suspension, with respect to the one or more API. This enables crystallinity to be maintained, which is particularly advantageous.

A pharmaceutical composition as described and claimed herein is suitable use as a medicament, as will be understood. For example, a pharmaceutical composition according to the invention for use in the treatment of a pulmonary condition in a patient may be provided. Suitably, such a composition may be administered via dry powder inhaler.

Where a dry powder inhaler is employed, this may for example be a single use inhaler. A suitable dry powder inhaler may comprise a mouthpiece, an inhaler body and a cartridge for receiving a dose comprising a pharmaceutical composition according to the invention described and claimed herein. For example, the cartridge may be moveable in relation to the inhaler body, for making the dose available through the mouthpiece. In one aspect, the cartridge may comprise one reservoir or multiple reservoirs. Each reservoir may, for example, provide a single dose.

It will be understood that the invention therefore also provides a dry powder inhaler comprising a pharmaceutical composition according to the invention as described and claimed herein.

In one aspect, the invention can be carried out by for example preparing a suspension of a coarse hydrophobic API in water and mixing until a homogenous suspension is obtained, followed by high shear mixing for a suitable period, for example 1 h. The one or more force control agents may also, if desired, be added at this stage, or may be added later as described below (or addition may be done at both stages). The force control agent if added at this stage can be mixed so as to obtain a solution of the force control agent in the antisolvent, with the API remaining in suspension. The particle size of the suspended API can be reduced by for example high pressure homogenization at a pressure of, for example, 50 bar for 20 cycles with a 200 μm chamber, plus 10 cycles with a 100 μm and 200 μm chambers, while ensuring a homogenous suspension. Thus, in one aspect of the invention, the API and one or more FCA may be subjected to the step of reducing the particle size distribution of the composition together. Alternatively, or in addition, the one or more FCA may be added after the step of reducing the particle size distribution of the composition. The high-pressure homogenization process reduces particle size by passing the liquid through a narrow gap under high pressure where the different processing parameters such as pressure, solid concentration and number of cycles lead to changes in particle size. A force control agent, for example, L-leucine, is added to the suspension and the mixture is stirred until full L-leucine dissolution. L-leucine can be added two times throughout the drying in order to target an L-leucine content of 0%, 5% and 15%. For each L-leucine addition, the suspension can be stirred for at least 30 min to ensure full dissolution. The API, L-leucine and water mixture is spray-dried while stirring with a feed rate of, for example, 10 g/min, an outlet drying temperature of, for example, 75° C., an inlet temperature of, for example, 135-150° C. and a rotameter set to, for example, 50 mm for the atomization flowrate, in an open-loop configuration. The spray drier can, for example, be equipped with a two fluid nozzle with a 2.2 mm cap and 1.5 mm orifice, and a high-performance cyclone. An amount, for example, about 30 mg of the resulting composition is filled to, for example, size 3 HPMC capsules which are ready to be actuated using a DPI device.

Error! Reference source not found. shows a schematic representation of the process comprising: (i) API particle size distribution reduction step in which the micronized API with target particle size distribution is obtained suspended in an anti-solvent system. (ii) Addition of a force control agent (FCA) soluble in the solvent system used, leading to a mixture of solvent, micronized API in suspension and dissolved FCA. (iii) Removal of the solvent from the mixture by spray drying, leading to coating of the micronized API with the dissolved FCA.

The step of particle size reduction carried out in step (i) of the process of the invention can be any suitable particle size reduction step. Such methods are known to the skilled person. Preferably, the size reduction step (i) is performed by high pressure homogenization, microfluidization, ball milling, high shear mixing, or any combination of thereof. Most preferably, the size reduction step (i) is performed by high pressure homogenization or microfluidization. Preferably, jet milling is not used. We have found this can be harsh, and potentially disadvantageous, such that some amorphous material may be produced. The process of the present invention, in particular using wet-milling techniques such as high-pressure homogenization or microfluidization, enables the crystallinity of the API to be preserved throughout the process.

The solvent system used is an anti-solvent of the at least one API and at least one excipient (the one or more FCA) dissolves or partially dissolves in it. The term “anti-solvent” as used herein describes a solvent or solvent system that a substance is substantially insoluble in, thus when a substance is mixed with its anti-solvent, the substance is suspended within the antisolvent as opposed to dissolving within in. Preferably, the term is used to refer to a solvent or solvent system in which said substance is completely insoluble. What is considered an anti-solvent for a particular substance is generally known to the skilled person. Typical solvents are for example water and ethanol, for processing one of more API with, for example, an FCA such as L-leucine. In these cases, the solvent in which the API is insoluble is water and ethanol. Other solvents include ketones, methylene chloride, methanol and other alcohols, such as polar protic solvents or alcohols, and are suitable for processing one of more API with, for example, DSPC.

Certain specific aspects and embodiments of the present invention will be explained in more detail with reference to the following examples. Example 1 is set forth to aid in understanding the invention but is not intended to, and should not be considered as to, limit its scope in any way.

Formulations in the examples of the present invention comprised one or more of the following materials:
Remdesivir
Leucine
Distearoylphophatidylcholine (DSPC)
Lecithin
Fluticasone Furoate

Formulations in the examples of the present invention were characterized by the following techniques:
A laser diffraction instrument was used for particle size distribution measurements for the suspension.
A laser diffraction instrument, combined with a Rodos dry dispersing unit and an Aspiros module (Sympatec GmbH, Germany) were used for particle size distribution measurements for the formulations. Dispersing pressures of 0.1 bar (using an R2 lens (0.45-87.5 μm), with a focal length of 50 mm) and pressures of 5 bar (for example 1), 4 bar (for example 2—Trial 1) and 2.5 bar (for example 2—Trial 2) (using an R1 lens (0.18-35 μm), with a focal length of 20 mm) were applied in order to determine the size of either agglomerates or single particles, respectively. The velocity was maintained at 50 mm/s.
Hydroxypropylmethyl cellulose (HPMC) size three capsules (Capsugel, Colmar, France) containing 30 mg±1.5 mg of powder were used for all in-vitro aerosolization studies. Example 1 materials were tested in the Next Generation Impactor (NGI) (Copley Scientific, Nottingham, UK) with a pre-separator connected to a vacuum pump (Copley Scientific, Nottingham, UK). The NGI cups were coated with 1 mL of 1% of glycerol in ethanol v/v) solution. 15 ml of dissolution media were placed in the pre-separator. Each test consisted of one actuation of the capsule into the NGI using either a 60 L/min or a 100 L/min DPI device, during 4 s or 2.4 s, respectively. API content deposited in each stage was recovered and analyzed by HPLC, enabling ED and FPD determination and the distribution among stages, assuring a mass balance of the recovered material with an error below 15%. All aerodynamic performance experiments were carried out in triplicate. Example 2 materials were tested by gravimetric Fast Screening Impactor (FSI). Each test consisted of one actuation of the capsule into the FSI equipped with USP Induction Port and Pre-Separator (Copley Scientific, Nottingham, UK) using a 100 L/min DPI device during 2.4 s. The FSI filter was weighed before and after capsule actuation and the fine particle dose (of API) was calculated by this difference and by correcting the amount of powder retained in the filter by the determined Assay (% w/w) value. The combined device and capsule was also weighed before and after actuation to determine the non-emitted fraction. The experiments were run in triplicate.
X-ray powder diffraction (XRPD) patterns were obtained with a PANalytical (Malvern, UK) X′Pert PRO X-ray diffraction system using Cu K radiation (λ=1.54 A°). The generator voltage and current intensity were set at 45 kV and 40 mA, respectively, and the 2 θ scanning range was from 4° to 40° with a step size 0.0131303° and a count time of 99.450 s per step. Samples were loaded using the zero-background technique.

A pharmaceutical composition comprising micronized coated API particles according to the present invention or obtained according to a process of the present invention can be used as a medicament in the treatment of a pulmonary condition in a patient. Such treatment can comprise administration via dry powder inhaler. The pharmaceutical composition of the present invention can be delivered by a dry powder inhaler such as a single use inhaler. The inhaler can comprise a mouthpiece, an inhaler body, and a cartridge for receiving the dose wherein cartridge can be moveable in relation to the inhaler body, for making the dose available through the mouthpiece. The inhaler cartridge comprises one reservoir or multiple reservoirs and each reservoir provides a single dose.

The present invention is of utility in enabling high dosages of API to be provided, particular via the inhalation route. Thus, the invention provides a pharmaceutical composition as described, wherein the composition is a high dosage inhalation composition wherein a single inhaled dose provides at least 2.5 mg of API or more, such as greater than 5 mg or more. High dosage can also refer to where the amount of API in the inhaled drug dose is above 4% by weight of the dose (see for example Sibum et al, Challenges for pulmonary delivery of high powder doses, International Journal of Pharmaceutics. 2018, 548:325-336. Doi: 10.1016/j.ijpharm.2018.07.008;
or Adhikari et al, Solid state of inhalable high dose powders, Advanced Drug Delivery Reviews. 2022, 189:114468. doi: 10.1016/j.addr.2022.114468.

### Example 1—Varying L-Leucine Content in Remdesivir after Wet Milling

Remdesivir (65 g) was suspended in water (802 g), and mixed until a uniform suspension was obtained, high shear mixed for 1 h and fed to a lab scale microfluidizer processor where the suspension was submitted to pressures of 50 bar for 20 cycles with a 200 μm chamber, plus 10 cycles with a 100 μm and 200 μm chambers. The following particle size results were obtained: Dv10=1.2 μm; Dv50=2.4 μm; Dv90=4.5 μm. After this particle size reduction step, the suspension was fed to a lab scale spray dryer (Buchi, model B-290) while stirring, with a feed rate of 10 g/min, an outlet drying temperature of 75° C., an inlet temperature of 135-150° C. and a rotameter set to 50 mm for the atomization flowrate, in an open-loop configuration. The spray drier was equipped with a two fluid nozzle with a 2.2 mm cap and 1.5 mm orifice, and a high-performance cyclone. Before feeding the solution to the nozzle, the spray drying unit was stabilized with nitrogen and then with solvent (water) to assure stable inlet and outlet temperatures.

L-leucine was added two times in this example in order to target an L-leucine content of 0% (Trial 1), 5% (Trial 2) and 15% (Trail 3). For each L-leucine addition, this was added to the Remdesivir suspension, and the suspension was stirred (prior to any spray drying) for at least 30 min to ensure full dissolution.

All spray-drying trials were characterized for crystalline state of API and L-leucine by XRPD, geometric particle size by Malvern laser diffraction with a suitable anti-solvent and for assay by HPLC. All capsule trials were characterized for assay by HPLC, for aerodynamic performance by a next generation impactor, with the amount deposited in each stage quantified by HPLC. All trial capsules were tested using a PowdAir device (60 L/min at 4 kPa). Trial 1 and 3 capsules were actuated using a RS01 Plastiape (100 L/min at 4 kPa). The results are presented in Table 1 and Table 2.

The spray-dried product presented an assay of 102.9% w/w, 97.3% w/w and 83.2% w/w, thus giving an L-leucine content of 2.7% w/w and 16.8% w/w, for Trials 2 and 3, calculated by difference. Additionally, the geometric particle size from the suspension was maintained upon spray-drying, with a slight decrease of Dv90 for trial 2 (Dv90=4.1 μm) possibly due to method variability or powder sampling. The XRPD results indicate the API is maintained as crystalline material (absence of amorphous halo) and that the L-leucine is crystalline (crystalline peaks present).

These results indicate the addition of different amounts of L-leucine to the suspension can be used to manufacture products with target compositions. Moreover, the addition of L-leucine does not have a significant impact on particle size distribution.

TABLE 1
Spray dried product characterization for example 2.

| | | |
|---|---|---|
| Trial 1 | Trial 2 | Trial 3 |

Suspension

| | | | |
|---|---|---|---|
| Dv10 (μm) | 1.2 | 1.2 | 1.2 |
| Dv50 (μm) | 2.4 | 2.4 | 2.4 |
| Dv90 (μm) | 4.5 | 4.5 | 4.5 |

Spray-dried material

| | | | |
|---|---|---|---|
| Dv10 (μm) | 1.3 | 1.3 | 1.3 |
| Dv50 (μm) | 2.4 | 2.3 | 2.4 |
| Dv90 (μm) | 4.4 | 4.1 | 4.3 |
| SPAN | 1.3 | 1.2 | 1.3 |
| XRPD | Conforms | Conforms to the | Conforms to the |
| | to the API | API reference | API reference |
| | reference | diffractogram with | diffractogram with |
| | diffractogram | L-leucine peaks | L-leucine peaks |
| | | (e.g. 17.5 theta) | (e.g. 17.5 theta) |
| Assay | 102.9 | 97.3 | 83.2 |
| (% w/w) | | | |
| Impurities | 0.11 | 0.11 | 0.15 |
| (% area) | | | |
| Leucine | 0 | 2.7 | 16.8 |
| content (%) | | | |

Following micronization and spray-drying, the powder was filled into HPMC size 3 capsules using an Auger filler Quantos equipment at 20-25° C. and 50±10% RH, targeting a fill-weight of 30 mg and a rejection limit of ±5%. The filled capsules were actuated using a PowdAir inhaler (flow rate of 60 L/min for a pressure drop of 4 kPa) and Plastiape inhaler (flow rate of 100 L/min for a pressure drop of 4 kPa).

The manufactured capsules were characterized for aerodynamic performance by NGI, summarized in Table 2.

For the PowdAir device (60 L/min flow rate), the filled capsules have a fine particle fraction of 6.9±1.8% of emitted dose for the API alone, and 43.0±9.2 and 37.9±2.9% of emitted dose, for Trial 2 and 3, containing L-leucine as a force control agent. These results indicated the present of L-leucine in a range of 2.7 to 16.8% leads to FPF improvement of more than 4 times, comparing with the API alone formulation, for remdesivir and the PowdAir device.

For the Plastiape device (100 L/min flow rate) the filled capsules have a fine particle fraction RSD of 76% for the API alone, and 2.1% for Trial 3, containing L-leucine as a force control agent. These results indicated the present of L-leucine in a range of 2.7 to 16.8% leads to a variability decrease of more than 30 times, comparing with the API alone formulation, for remdesivir and the Plastiape device, as L-leucine acts to make more uniform the particle-particle and particle-device interactions (for example, a decrease of energy “hot-spots” of micronized particles, and decrease of agglomerates).

TABLE 2
Summary of the capsule characterization for example 1.

| | Trial 1 | | Trial 2 | Trial 3 | |
|---|---|---|---|---|---|
| Fill weight (mg) | 30 ± 1 | | 30 ± 1 | 30 ± 1 | |
| Label claim (mg) | 30 | | 28.5 | 25.5 | |
| Assay (% w/w) | — | | 98.2 | 83.8 | |
| Device | PowdAir | Plastiape | PowdAir | PowdAir | Plastiape |
| | (60 L/min) | (100 L/min) | (60 L/min) | (60 L/min) | (100 L/min) |
| FPD (mg/caps) | 0.4 ± 0.4 | 10.7 ± 7.4 | 6.6 ± 4.3 | 6.9 ± 0.8 | 10.2 ± 2.8 |
| FPD RSD (%) | 89.5 | 85.1 | 65.5 | 11.9 | 28.0 |
| EDNGI (mg/caps) | 6.9 ± 8.7 | 16.3 ± 9.2 | 15.0 ± 9.4 | 18.6 ± 3.3 | 15.9 ± 4.2 |
| EDNGI RSD (%) | 126 | 56 | 63 | 18 | 26 |
| MMAD (μm) | 2.7 | 2.8 | 2.4 | 3.4 | 2.9 |
| GSD | 2.7 | 2.3 | 1.9 | 2.1 | 2.0 |
| FPFED, NGI (%) | 6.9 ± 1.8 | 48.3 ± 30.0 | 43.0 ± 9.2 | 37.9 ± 2.9 | 64.1 ± 1.4 |
| FPFED, NGI RSD (%) | 26.0 | 76.2 | 21.3 | 7.6 | 2.1 |

FPD—fine particle dose; RSD—relative standard deviation; EDNGI—emitted dose determined by next generation impactor; NGI—next generation impactor; MMAD—mass median aerodynamic diameter; GSD—geometric standard deviation; FPFED—fine particle fraction of the emitted dose.

### Example 2—Varying FCA in Fluticasone Furoate after Wet Milling

Fluticasone Furoate was suspended in water and micronized by a microfluidizer until the target particle size was obtained. After this particle size reduction step, the force control agent (Lecithin or DSPC) was dissolved in the suspension (formulations described in Table 3). The suspension was then fed to a lab scale spray dryer (Buchi, model B-290) while stirring, with a feed rate of 6 g/min, an outlet drying temperature of 50° C., an inlet temperature of 82-91° C. and a rotameter set to 40 mm for the atomization flowrate, in an open-loop configuration. The spray drier was equipped with a two fluid nozzle with a 1.4 mm cap and 0.7 mm orifice, and a high-performance cyclone. Before feeding the solution to the nozzle, the spray drying unit was stabilized with nitrogen and then with solvent (water) to assure stable inlet and outlet temperatures.

All spray-drying trials were characterized for crystalline state of API, geometric particle size by dry dispersion (Sympatec) and for assay by HPLC. All capsule trials were characterized for assay by HPLC, for aerodynamic performance by a fast-screening impactor (FSI), with the amount deposited in each stage quantified by weight. All trial capsules were tested using a RS01 Plastiape device (100 L/min at 4 kPa). The results are presented in Table 4 and Table 5.

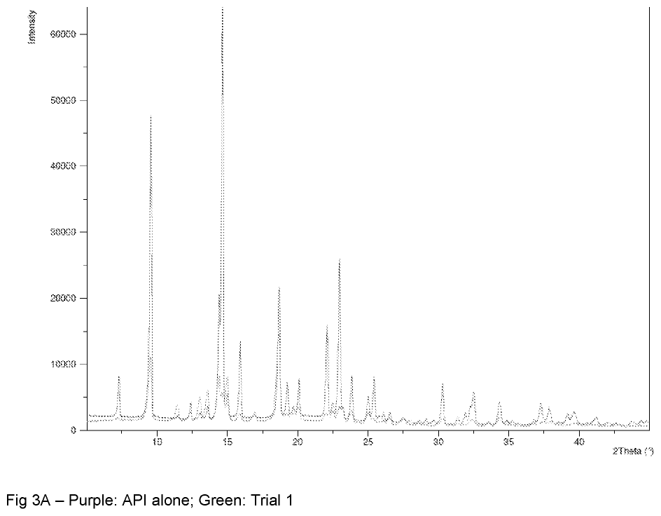

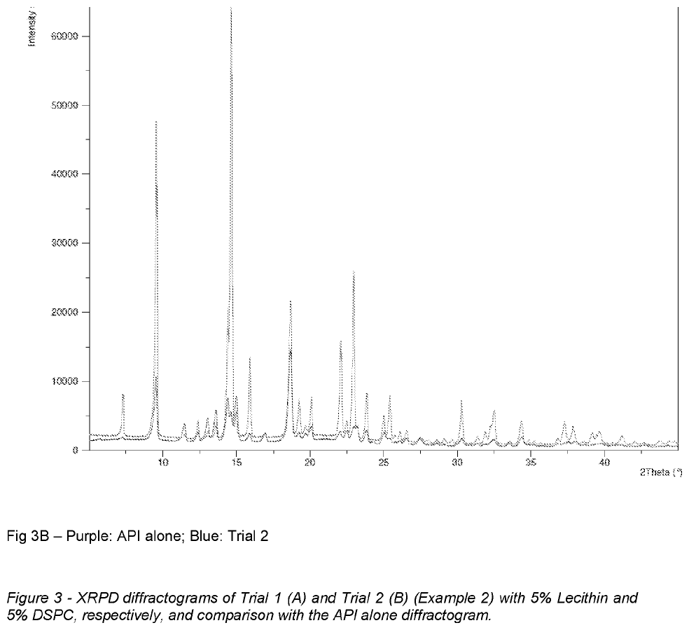

The spray-dried product presented an assay of 93.6% w/w and 93.4% w/w. The XRPD results (FIG. 3) indicate the API is maintained as crystalline material (absence of amorphous halo).

TABLE 3
Formulations for example 2.

| | Example 2 -Trial 1 | Example 2 - Trial 2 |
|---|---|---|
| Fluticasone Furoate (% w/w) | 95.0 | 95.0 |
| Lecithin (% w/w) | 5.0 | |
| DSPC (% w/w) | | 5.0 |
| TOTAL | 100.0 | 100.0 |

TABLE 4
Spray dried product characterization for example 2.

| | Example 2 - Trial 1 | Example 2 - Trial 2 |
|---|---|---|
| Dv10 (μm) | 0.57 | 0.59 |
| Dv50 (μm) | 1.66 | 1.82 |
| Dv90 (μm) | 4.27 | 5.63 |
| SPAN | 2.2 | 2.8 |
| XRPD | Conforms to the API | Conforms to the API |
| | reference diffractogram | reference diffractogram |
| Assay (% w/w) | 93.6 | 93.4 |

*ND: Not determined

Following micronization and spray-drying, the powder was filled into HPMC size 3 capsules using an Auger filler Quantos equipment at 20-25° C. and 40±10% RH, targeting a fill-weight of 30 mg and a rejection limit of ±7%. The API alone was also filled into capsules for comparison purposes. The filled capsules were actuated using a Plastiape inhaler (flow rate of 100 L/min for a pressure drop of 4 kPa). The manufactured capsules were characterized for aerodynamic performance by FSI, summarized in Table 2.

From the results presented in Table 4, both the FPD and the FPF are improved by adding a force control agent such as Lecithin or DSPC. When comparing the API alone results with Trials 1 and 2, we can observe at least a 2-fold increment in FPD, promoted by the addition of the force control agent.

TABLE 5
Summary of the capsule characterization for example 2.

| | API alone | Trial 1 | Trial 3 |
|---|---|---|---|
| Fill weight (mg) | 30 ± 2 | 30 ± 2 | 30 ± 2 |
| Label claim (mg) | 30 | 28.5 | 28.5 |
| Assay (% w/w) | — | 93.6 | 93.4 |
| Device | Plastiape | Plastiape | Plastiape |
| | (100 L/min) | (100 L/min) | (100 L/min) |
| FPD (mg/caps) | 3.5 ± 0.1 | 6.9 ± 0.1 | 9.9 ± 0.4 |
| FPD RSD (%) | 2.9 | 1.4 | 4.0 |
| EDFSI (mg/caps) | 13.1 ± 2.4 | 20.8 ± 0.6 | 14.30 ± 0.6 |
| EDFSI RSD (%) | 18.3 | 2.9 | 4.2 |
| FPFED, NGI (%) | 27.1 ± 4.7 | 33.3 ± 0.5 | 69.5 ± 4.6 |
| FPFED, NGI RSD (%) | 17.3 | 1.5 | 6.6 |

FPD—fine particle dose; RSD—relative standard deviation; EDFSI—emitted dose determined by fast screening impactor; FSI—fast screening impactor; FPFED—fine particle fraction of the emitted dose.

1-41. (canceled)

42. A pharmaceutical composition suitable for a dry powder inhaler, which composition comprises particles comprising one or more micronized crystalline active pharmaceutical ingredients (API) coated with one or more force control agents (FCA), wherein the coated particles are obtained by addition of the said one or more force control agents (FCA) to a wet-milled crystalline suspension of the API prior to spray drying, and wherein one or more API consists of crystalline API.

43. A pharmaceutical composition according to claim 42 wherein the wet-milled crystalline suspension of API is wet-milled by microfluidization or by high-pressure homogenization.

44. A pharmaceutical composition according to claim 42 wherein the particle size range has a Dv90 of <10 μm, as measured by laser diffraction.

45. A pharmaceutical composition according to claim 44 wherein the particle size range has a Dv90 of <6 μm, as measured by laser diffraction.

46. A pharmaceutical composition according to claim 42 wherein the particles comprising said micronized crystalline one or more API coated with one or more FCA have a higher fine particle fraction (FPF) when compared with a pharmaceutical composition comprising the same micronized particles but without any FCA, and
wherein the fine particle fraction (FPF) is 30% or more of the emitted dose, when testing a capsule comprising the said composition in a dry powder inhaler.

47. A pharmaceutical composition according to claim 42, wherein the one or more FCA are chosen from the group comprising: leucine (i.e. L-leucine), isoleucine, tri-leucine, distearoylphophatidylcholine (DSPC), dipalmitoylphosphatidylcholine (DPPC), alanine, lecithin, arginine, histidine, lysine, valine, or magnesium stearate.

48. A pharmaceutical composition according to claim 42, wherein the one or more API are chosen from the group comprising crystalline API insoluble in a solvent in which the FCA is soluble.

49. A pharmaceutical composition according to claim 42, wherein the one or more FCA is present in a concentration of 30% w FCA/w API or less.

50. A process for manufacturing a pharmaceutical composition suitable for a dry powder inhaler according to claim 42, which process comprises the steps of:
a. Reducing the particle size distribution of the composition to obtain the micronized crystalline one or more API with a target particle size distribution suspended in an antisolvent system;
b. Adding one or more FCA soluble in the said antisolvent system either before or after, or both before and after, said reducing step, so as to provide a mixture of antisolvent with FCA dissolved therein, and micronized API in suspension;
c. Removal of the antisolvent from the mixture by spray drying, so as to obtain coating of the micronized crystalline API with the dissolved FCA;
wherein the particle size distribution reduction step comprises subjecting the particles to High Pressure Homogenization, microfluidization, ball milling, high shear mixing, or any combination thereof.

51. A process according to claim 50, wherein the particle size distribution reduction step comprises microfluidization using a solvent system in which the one or more API is insoluble.

52. A process according to claim 50, wherein the particle size distribution reduction step comprises High Pressure Homogenization using a solvent system in which the one or more API is insoluble.

53. A process according to claim 50, wherein the temperature in the particle size distribution reduction step is below 60° C.

54. A process according to claim 50, wherein the pressure in the particle size distribution reduction step is 100 bar or greater.

55. A process according to claim 50, wherein the pressure in the particle size distribution reduction step is below 100 bar.

56. A process according to claim 50, wherein the antisolvent system comprises only one antisolvent for steps (a), (b) and (c).

57. A process according to claim 50, wherein the antisolvent system is chosen from the group comprising: water, ethanol, methylene chloride, methanol, and other alcohols, such as a C3, C4 or C5 aliphatic alcohol; a ketone; and polar protic solvents.

58. A process according to claim 50, wherein the total solids content in the antisolvent system is below 20% by weight, preferably below 15% by weight, most preferably below 10% by weight.

59. A pharmaceutical composition comprising micronized coated API particles obtained or obtainable by a process according to claim 50.

60. A pharmaceutical composition for use in the treatment of a pulmonary condition in a patient, comprising administration via dry powder inhaler, wherein the dry powder inhaler comprises a mouthpiece, an inhaler body, and a cartridge for receiving a dose comprising a pharmaceutical composition according to claim 42.

61. A dry powder inhaler comprising a pharmaceutical composition according to claim 42.
